(19) United States
(12) Patent Application Publication
(10) Pub. No.: US 2010/0113939 A1
(43) Pub. Date: May 6, 2010

(54) SMART BALLOON CATHETER

(76) Inventors: Hiroshi Mashimo, Lincoln, MA (US); Raj K. Goyal, Newton, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/311,507

(22) PCT Filed: Oct. 1, 2007
(86) PCT No.: PCT/US07/21110

§ 371 Date: Jan. 4, 2010

Related U.S. Application Data
(60) Provisional application No. 60/827,729, filed on Oct. 2, 2006.

(51) Int. Cl. A61B 5/0215; A61M 25/10; A61B 8/12

(52) U.S. Cl. 600/470; 600/488; 606/194

(57) ABSTRACT

The invention provides techniques for the diagnosis and treatment of a narrowing lumen with a smart balloon catheter. The smart balloon catheter includes pressure and diameter sensing features along with a feedback system to control the dilation of the balloon. Ambient pressure of the lumen is detected with multiple pressure sensors located on the distal end of the catheter and displayed on a monitoring device. Ambient pressure results are used to position the distal end of the catheter within the narrowing lumen. A controlled gradual, or stepwise, dilation of the balloon occurs. The pressure sensors detect the ambient pressure of the lumen outside the of the balloon, and the pressure within the balloon. Distances sensors measure the distance between the center of the catheter and the expanded balloon surface. The diameter of the balloon at different cross-sections is determined and displayed on the monitoring device. The volume of the balloon, and the waist of the narrowing lumen, are determined. The rate of the dilation continues as a function of input provided by pressure and distance sensors. the dilation halts based on pressure, distance or volume endpoints.

### BACKGROUND

Modern techniques for the diagnosis and treatment of narrowing of lumen (i.e. hollow tubes in the body) can use dilating balloon catheters. For example, angioplasty catherization typically involves locating an obstruction in a lumen and then inserting a balloon catheter into the lumen at the location of the obstruction. Once the balloon is positioned within the obstruction, it is dilated with a fluid causing the balloon to expand against the obstruction. A similar process has also been used for the sweeping of ducts (such as the removal of concretions including gallstones and kidney stones). A common risk, however, with both procedures is the possibility of improper dilation of the balloon portion of the catheter. In the case of under-dilation, the effect of the catherization on the obstruction may be insufficient and therefore require additional treatments, adding to procedure time and increasing the risk of potential complications. In the case of over-dilation, the over-expanded balloon catheter can cause cracks in the lumen, or in the extreme, may cause an aneurysm or tear in the lumen.

In general, the rate and extent to which a balloon catheter may be dilated is based in part on the nature of the obstruction and the type of lumen being treated. For example, an esophogeal web is a mucosal defect that can be dilated very rapidly and easily, while a mucosal fibrosis as noted in common acid reflux-associated strictures generally require greater dilation pressures and volumes. Moreover, a neuromuscular defect, as noted in muscular rings and achalasia, are more compliant than fibrotic strictures but require volumes well-beyond those of the lumen to disrupt the muscle fibers. This variation in constriction responses associated with the nature of an obstruction and the type of lumen highlights the importance of control over dilation set-points such as the rate of dilation, pressure, volume and the diameter of the balloon dilator. Present methods of dilation, however, are largely blind procedures and often involve passage of stiff tapered rods or use balloons which do not have sensors within the balloon dilator for assessing information regarding pressure, volume or diameter.

### SUMMARY

In general, in an aspect, the invention provides a catheter system including a distal pressure transducer, a proximal pressure transducer, an expandable membrane located between the distal pressure transducer and the proximal pressure transducer, and a membrane pressure transducer disposed on the catheter within an area enclosed by the membrane, wherein the distal and proximal pressure transducers are configured to measure pressure outside of the membrane, and the membrane pressure transducer is configured to measure pressure inside of the membrane.

Implementations of the invention may include one or more of the following features. The catheter includes at least one barostat port disposed within the membrane. The catheter includes at least one radio-opaque marker configured to expand and contract with the membrane. The catheter includes at least one pair of ultrasonic crystals affixed to the membrane.

In general, in another aspect, the invention provides a catheter for the diagnosis and treatment of a narrowing lumen, including a plurality of pressure transducers disposed within the catheter and arranged proximally from substantially near the distal end of the catheter, at least one expandable membrane located over at least one pressure transducer, at least one dilation source configured to expand the at least one membrane with a fluid, and three pairs of electrode rings disposed proximally, center, and distally within the at least one membrane, such that the electrode rings are configured to detect the diameter of the balloon membrane.

Implementations of the invention may include one or more of the following features. The catheter fluid is isotonic saline. The catheter pressure transducers include thermoplastic elastomers (TPE).

In general, in another aspect, the invention provides a method for the diagnosis and assessment of a narrowing lumen with a balloon catheter, such that the balloon catheter includes a plurality of pressure transducers, a balloon membrane, and a plurality of ring electrodes configured to detect the diameter of the lumen and the balloon membrane, the method including detecting ambient pressure within the lumen, positioning the balloon catheter within the lumen, dilating the balloon membrane, and detecting the diameter of the balloon membrane.

Implementations of the invention may include one or more of the following features. The method further includes detecting the diameter of the lumen. The method further includes executing a rapid dilation algorithm to perform a rapid bougienage on a mucosal stricture within the lumen. The method further includes executing a gradual dilation algorithm for muscular disorders within the lumen. Detecting the diameter of the balloon membrane includes detecting a plurality of diameters along the length of the balloon membrane. The method further includes terminating the dilation of the balloon catheter based on the equalization of diameters along the balloon membrane. Positioning of the balloon catheter within the lumen is based on the ambient pressure within the lumen. The method further includes detecting the pressure within the balloon membrane. The method further includes establishing an end-point for dilation of the balloon catheter based the diameter of the balloon membrane. The method further includes sweeping a concretion with the dilated balloon membrane.

In general, in another aspect, the invention provides a catheter including a balloon membrane, inflation means coupled to the balloon membrane for controlled inflation of the balloon membrane, sensing means disposed within the balloon catheter for simultaneously detecting the diameter of the balloon membrane and the pressure within the balloon membrane, and processing means coupled to the inflation means and the sensing means for determining a rate of the controlled inflation of the balloon membrane.

Implementations of the invention may include one or more of the following features. The processing means is further configured for determining an end-point for the inflation of the balloon membrane. The processing means is further configured to determine the rate of the controlled inflation with a closed-loop proportional control algorithm.

In general, in another aspect, the invention provides a catheter including a balloon membrane, at least one pair of ultrasonic crystals attached to the balloon membrane, an ultrasound generator that is operably connected to the ultrasonic crystals and that is configured to determine the diameter of the balloon membrane, a pressure transducer disposed on the balloon catheter and that is configured to sense the pressure within the balloon membrane, and a radio-opaque marker disposed on the balloon membrane.

Implementations of the invention may include one or more of the following features. The balloon membrane is comprised of an elastic polymer. The balloon membrane is a non-compliant balloon. The balloon membrane is a compliant balloon.

In accordance with implementations of the invention, one or more of the following capabilities may be provided: diagnosis and characterization of narrowing in a lumen; therapeutic dilation and sweeping of ducts; manometric positioning and correction of a catheter in a lumen; controlled stepwise dilation of a balloon catheter, simultaneous pressure and distance monitoring of a lumen during dilation; proportional and integral control of the dilation rate; and, control of lumen compliance dilation endpoints based on the etiology of the lumen, and pressure, diameter and volume of the inflatable membrane. A benefit of the controlled dilation and corresponding sensor feedback is to determine the physiological characteristics of the lumen wall (i.e., pressure and volume/diameter compliance), thereby helping to distinguish between different narrowing lumen etiologies and guide the appropriate management of the narrowing.

These and other features of the invention, along with the invention itself, will be more fully understood after a review of the following figures, detailed description, and claims.

### BRIEF DESCRIPTION OF THE FIGURES

FIG. 1 is a simplified diagram of a smart catheter system including components for dilation and monitoring a smart catheter.

FIG. 2 is an enlarged diagram of the distal end of a smart catheter with three pairs of electrode rings for performing impedance planimetry.

FIG. 3a is an enlarged view of the distal end of a smart catheter detecting the waist of an obstruction within a lumen.

FIG. 3b is an enlarged view of the distal end of a smart catheter sweeping a concretion.

FIG. 4 is a graph of pressure versus volume with representative compliance curves for different lumen etiologies.

FIG. 5 is an enlarged diagram of the distal end of a smart catheter with expandable radio-opaque markers.

FIG. 6 is an enlarged diagram of the distal end of a smart catheter with ultrasonic crystals.

FIG. 7 is a block flow diagram of a process for step-wise dilation of a smart balloon catheter.

### DETAILED DESCRIPTION OF PREFERRED EMBODIMENTS

Embodiments of the invention provide techniques for the diagnosis and treatment of a narrowing lumen with a smart balloon catheter. The smart balloon catheter includes pressure and diameter sensing features along with a feedback system to control the dilation of the balloon. The smart balloon catheter is placed within the narrowing lumen. The ambient pressure of the lumen is detected with multiple pressure sensors located on the distal end of the catheter. The pressure data is displayed on a monitoring device. Ambient pressure results are used to position the distal end of the catheter within the narrowing lumen. A controlled gradual, or stepwise, dilation of the balloon commences. The pressure sensors located outside of the balloon area on the catheter detect the ambient pressure of the lumen outside the of the balloon. Pressure sensors located inside the balloon area detect the pressure within the balloon. Distances sensors measure the distance between the center of the catheter and the expanded balloon surface. The diameter of the balloon at different cross-sections is determined and displayed on the monitoring device. The volume of the balloon is computed. The waist of the narrowing lumen is determined. The rate of the dilation continues as a function of input provided by pressure and distance sensors. The dilation halts based on pressure, distance or volume endpoints. This technique for the diagnosis and treatment of a narrowing lumen with a smart balloon catheter is exemplary, however, and not limiting of the invention as other implementations in accordance with the disclosure are possible.

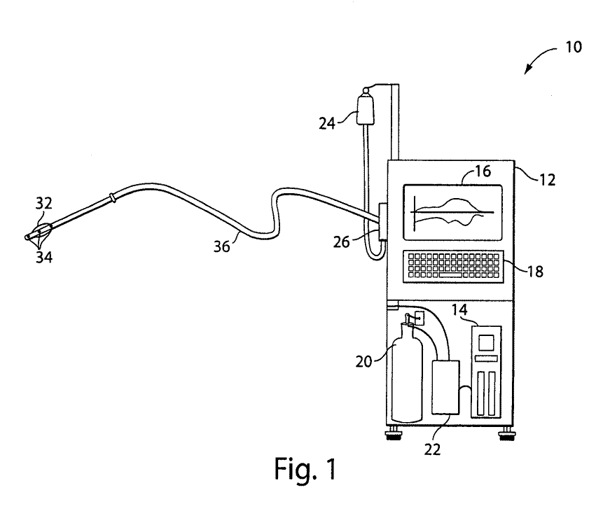

Referring to FIG. 1, a smart balloon catheter system 10 comprises a portable cabinet 12 including a computer 14, a monitor 16, a keyboard 18, a fluid reservoir 20, a pumping system 22, a drip bag 24, an interface junction 26, and a balloon catheter assembly 30 including an inflatable membrane 32, and pressure sensors 34. The portable cabinet 12 is configured to be transported from treatment area to treatment area and may receive external power via a standard facility outlet. The portable cabinet 12 may also include power conditioning components (e.g., UPS, line filters, and battery back-up). The computer 14, monitor 16 and keyboard 18 are operably connected and configured to provide a user access to the memory and processing capabilities of the computer 14. In one embodiment, the computer 14 is a standard Personal Computer including associated memory devices, processors and operating software. The fluid reservoir 20 is configured to provide a fluid to the pumping system 22. The fluid reservoir 20 may include a liquid such as water or saline, or may also include compressed gases such as oxygen or Clean Dry Air. The pumping system 22 is operably connected to the fluid reservoir 20, the computer 14 and the interface junction 26. The pumping system 22 includes pressure and flow sensors and is configured to receive pumping commands from the computer 14. For example, the pumping system 22 includes, but is not limited to barostats or step-wise syringe pumps. The smart balloon system 10 optionally includes a drip bag 24 that is operably connected to the interface junction 26 and configured to provide a flushing fluid to the distal end of the smart balloon catheter. The interface junction 26 is operably connected to the proximal end of the smart balloon catheter 30, the computer 14 and the pumping system 22, and is configured to direct fluid to and from the smart balloon catheter 30, as well as transfer sensor information from the smart balloon catheter 30 to the computer 14. The pumping system 22 is also configured to supply the fluid in a series of constant volume units to provide a step-Wise dilation capability. The smart balloon catheter system 10 is configured to transmit fluid to and from the interface junction 26 to the inflatable membrane 32, and signal information from the sensors 34 to the computer 14, via the interface junction 26. The distal end of the smart balloon catheter 30 is inserted into a patient for the diagnosis and treatments of narrowing of lumen, i.e., hollow tubes of the body, including by not limited to, narrowing of cardiovascular, gastrointestinal, biliary, pancreatic, respiratory, and genitourinary tracts, ducts, tubes and vessels including stenotic, fibrotic, atheromatous, atretic, cystic, malignant, post-surgical, post-ischemic, post-irradiation, anastomotic, traumatic, muscular, mucosal, acquired and congenital narrowings. The smart balloon catheter 30 may also be configured to sweep ducts (e.g., for the removal of concretions including gallstones and kidney stones) and can be applied to a variety of pathologies of the lumen including those of coronary arteries, cardiac valves, arteries and veins, oropharynx, esophagus, stomach, pylorus, intestine, colon, rectum, anus, bile, and pancreatic ducts, trachea, bronchi, fallopian tube, uterus, vagina, urethra, ureter, penis, and various anastomoses. The catheter system 10 can also be passed through existing biopsy or therapeutic channels of standard endoscopes for visualizing many of these lumen.

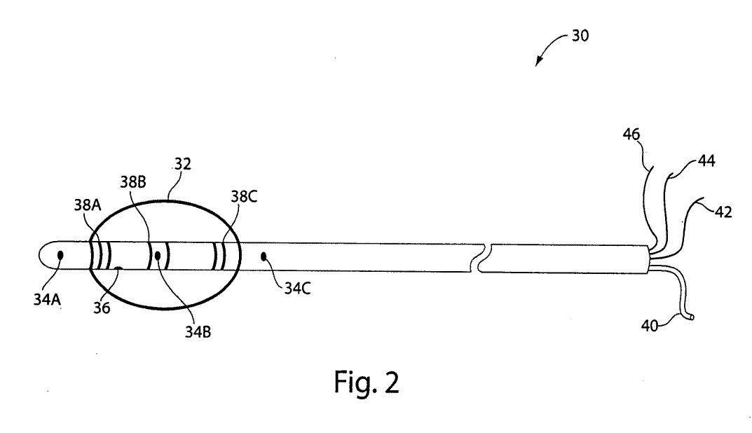

Referring to FIG. 2, with further references to FIG. 1, the distal end of the smart balloon catheter 30 includes the inflatable membrane 32, three pressure sensors 34a, 34b, 34c, balloon fluid infusion port 36, three pairs of impedance planimeters 38a, 38b, 38c, a fluid transport tube 40, impedance planimeter control connections 42, electric field control connections 44, and transducer control connections 46. In general, the smart balloon catheter 30 is comprised of material of either high or low compliance depending on the application, and is dimensioned appropriately for the lumen under diagnosis or treatment. The inflatable membrane 32 is connected to the distal end of the smart balloon catheter 30 and is configured to expand and contract based on the volume and pressure of fluid provided by the fluid pump 22 through the port 36. The inflatable membrane 32 may be of differing; material, compliance, size and shape depending on the usage. For example, for the treatment of strictures with minimal fibrosis and high compliance, such as a mucosal web or stricture, a high compliance balloon with wide range of diameters can be used such as thin latex. On the other hand, full-thickness mucosal and muscular fibrosis as found at surgical anastomosis or transural fibrosis noted in Crohn's diseases of the gut can use less elastic/compliant materials similar to those used in CRE dilators. The inflatable membrane 32 is affixed to the catheter 30 and is configured to inhibit the flow of fluid provided through port 36 from leaving the confinement created within the space of the membrane 32. The pressure sensor 34b is disposed and configured to sense the pressure within the inflatable membrane 32. The distal pressure transducer 34a and the proximal pressure transducer 34c are disposed outside of the inflatable membrane 32, and are configured to sense the distal and proximal ambient pressure of the lumen in the area around the inflatable membrane 32. The pressure transducers 34a, 34b, 34c may be composed of thermoplastic elastomers (TPE), or a more traditional liquid or air filled sensor. The configuration of the pressure sensors 34a, 34b, 34c is exemplary and not limiting as additional sensors may be arranged along the length of the of the smart balloon catheter 30, and may be comprised of other materials.

The impedance planimeters 38a, 38b, 38c are disposed on the distal end of the smart balloon catheter 30 within the inflatable membrane 32, and are operably connected to the computer 14 via the interface junction 26 and the impedance planimeter control connection 42 and the electric field control connection 44. The impedance planimeters 38a, 38b, 38c are disposed proximally, center, and distally within the inflatable membrane, and configured to determine the radial distance from the catheter where the electrodes are placed to the wall of the balloon during inflation. For example, the impedance planimeters 38a, 38b, 38c will measure the diameter of inflatable membrane 30 during dilation. Three distance measurements will correspond with the distal, center, and proximal location of each set of impedance planimeters 38a, 38b, 38c. Impedance measurements correlate with diameter of the balloon at the midpoint between each pair of electrodes as this is proportional to the drop in impedance as saline or other semi-conducting fluid is introduced progressively into the balloon to inflate it. Additional pairs of impedance electrodes can be placed along the balloon for other applications requiring more accurate assessment of diameters along a longer tubular balloon, for example.

The fluid transport tube 40 is operably connected to the fluid infusion port 36 and the interface junction 26, and configured to direct fluid flow (e.g., saline, filtered air) into the expandable membrane 32. The fluid transport tube 40 is capable of providing positive and negative pressure to the membrane 32 during inflation and deflation respectively. The transducer control connections 46 are placed within the smart balloon catheter, and is operably connected to each pressure transducer 34a, 34b, 34c.

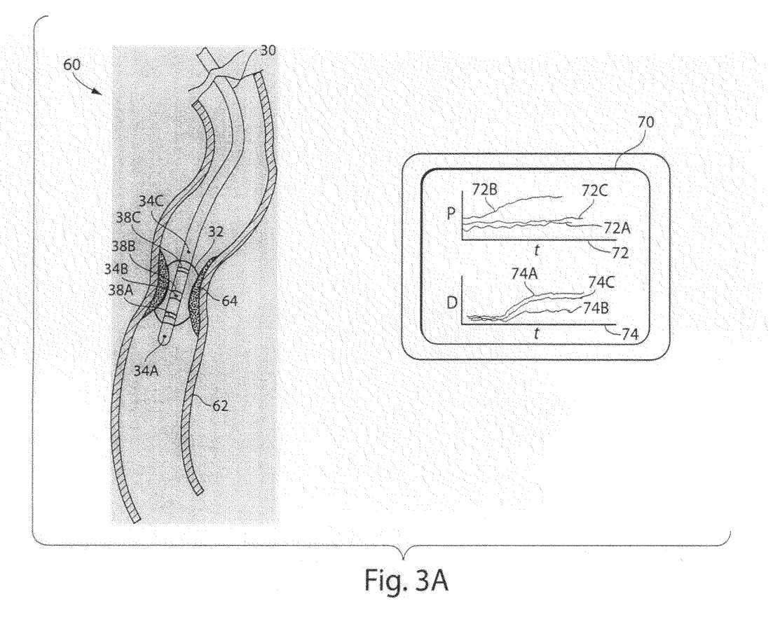

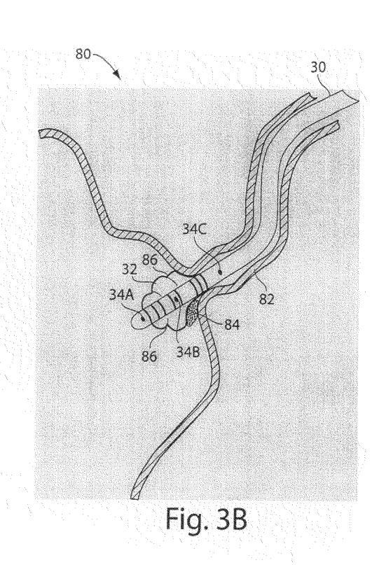

Referring to FIG. 3a, with further reference to FIGS. 1 and 3, an exemplary dilation of a narrowing lumen is depicted 60. The smart balloon catheter 30 is inserted into a narrowing lumen 62, and positioned at the site of an objection 64. The monitor 16 displays a graphic interpretation 70 of the pressure and distance readings 72, 74. The pressure transducers 34a, 34b, 34c provide manometric readings during the insertion of the smart balloon catheter 30. The pressure readings 72a, 72b, 72c, assist in the positioning of the distal end of the catheter 30 in relation to the lumen obstruction 64. This positioning can be confirmed with the aid of the fluoroscopic markers on the balloon. The distal and proximal pressure transducers 34a, 34c may also be used to monitor pressure changes in the lumen 62 not related to the narrowing or obstruction 64 (e.g., from respiration, cardiac pulsations, and abdominal pressures). During the step-wise dilation of the inflatable membrane 32, the distance the membrane is dilated is detected by the impedance planimeters 38a, 38b, 38c. The distance measurement for each set of planimeter rings 38a, 38b, 38c are displayed 74a, 74b, 74c. In addition to providing compliance analysis of the obstruction 64, the pressure and distance graphs 72, 74 enable an operator to determine the waist of the obstruction 64. The graphical representations 72, 74 of the pressure and distance date or exemplary only, and not a limitation, as other graphical representations may be provided by the computer 14 on the monitor 16.

Referring to FIG. 3b, with further reference to FIGS. 1 and 2, an exemplary sweeping of a duct is depicted 80. The smart balloon catheter 30 is inserted into a duct 82 and the inflatable membrane 32 is dilated. The membrane 32 can be configured with ridges 86 to assist in dislodging a concretion 84. In operation, the proximal pressure sensor 34c senses the ambient pressure within the duct 82. The center pressure sensor 34b senses the pressure within the inflatable membrane 32. The concretion 84 creates a temporary obstruction within the duct 82, which may be detected on the center pressure sensor 34b. The pressure within the membrane 32 is monitored in an effort to reduce the stress on the duct 82 during the sweep of the concretion 84. The pressure readings from the distal, center, and proximal sensors 34a, 34b, 34c are compared during the sweep, and the pressure within the membrane 32 is adjusted accordingly. For example, the computer 14 includes instructions to continuously monitor the pressure sensors 34a, 34b, 34c. If the center pressure sensor 34b indicates a high level, the computer 14 is further configured to instruct the fluid pumping system 22 to reduce the volume of fluid within the membrane 32. The ridges on the membrane 32 are exemplary only and not a limitation as other materials; compliance, sizes and shapes can be used (e.g., more tubular and elongate balloons with lower compliance can be used to diagnose and treat such lumens as the esophagus, intestines, and vessels where the narrowing may involve a longer span).

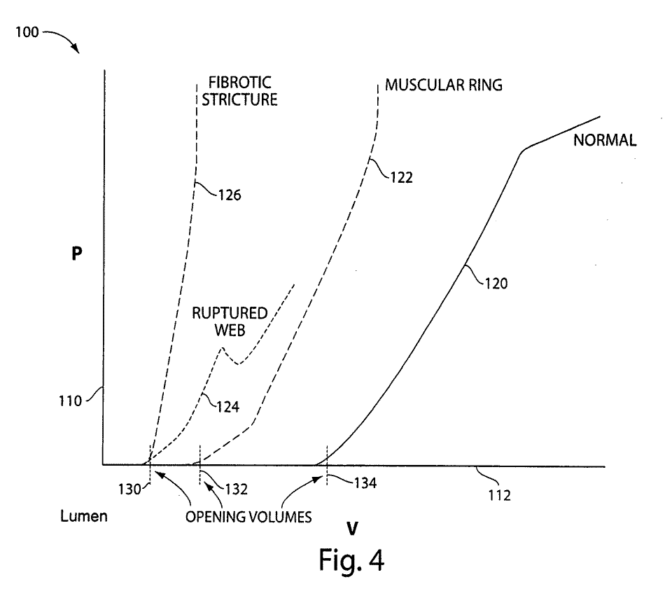

Referring to FIG. 4, an exemplary pressure versus volume graph 100 includes pressure units along the y-axis 110, volume units along the x-axis 112, and relative compliance curves for different lumen etiologies: normal 120, muscular ring 122, ruptured web 124, and fibrotic stricture 126. These compliance curves 120, 122, 124, 126 generally illustrate the resistance to dilation for each lumen example from a known opening volume 130, 132, 134. In general, the lumen compliance curves 120, 122, 124, 126 aid in the diagnosis and treatment of a narrowing lumen. For example, the etiology of lumen narrowing may be difficult to distinguish due to similarities in radiological and endoscopic appearances. The treatments for these different lumen etiologies, however, are quite different. In the esophagus, a web 124 is a mucosal defect that can be dilated rapidly by a variety of methods (e.g., Maloney, Savary, optical, or various balloon dilators). Peptic and non-peptic (e.g., ischemic, traumatic, post-surgical, post-irradiation) strictures 126 are associated with transmucosal fibrosis which require sufficient or more aggressive dilation to break a less compliant lesion. These methods, however, are generally less effective for muscular defects, or disorders of muscle relaxation such as achalasia or muscular rings 122, which may require slower dilations with larger diameters.

Physiological characteristics of the lumen wall measuring pressure and volume/diameter (compliance), as compared to the normal compliance 120, assist in distinguishing these etiologies, and guide in the appropriate management of the constricted area. In operation, the smart balloon catheter system 10 performs an appropriate controlled step-wise dilation for the particular lumen etiology for both diagnosis and treatment. For diagnosing a narrowing of lumen, the controlled step-wise dilation and corresponding pressure and distance feedback provide compliance data (e.g., pressure versus volume) to better classify the narrowing lumen. During treatment, the speed of dilation and the ultimate diameter of the dilation are controlled to assess the effectiveness of the dilation, rupture or sweeping treatment, and to avoid unnecessary injury to the affected tissue. Moreover, the balloon can be expanded to various sizes and pressure so that repeated passage and positioning of different sized dilators or balloons is not necessary. Also, simultaneous reading of the proximal and distal diameters of the balloon compared to the center can assess the efficacy of the dilation during the dilation—the electronic equivalent of assessing the “waist” of the balloon but without the need for fluoroscopy.

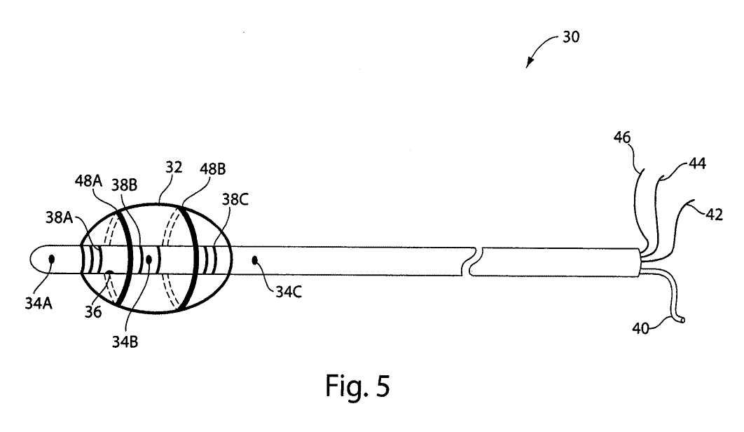

Referring to FIG. 5, with further reference to FIGS. 1 and 2, the distal end of the smart balloon catheter 30 includes radio-opaque markers 48a, 48b configured to expand and contract with the inflatable membrane 32. The radio-opaque markers 48a, 48b increase the resolution of the membrane during fluoroscopic viewing when the catheter is disposed within a lumen.

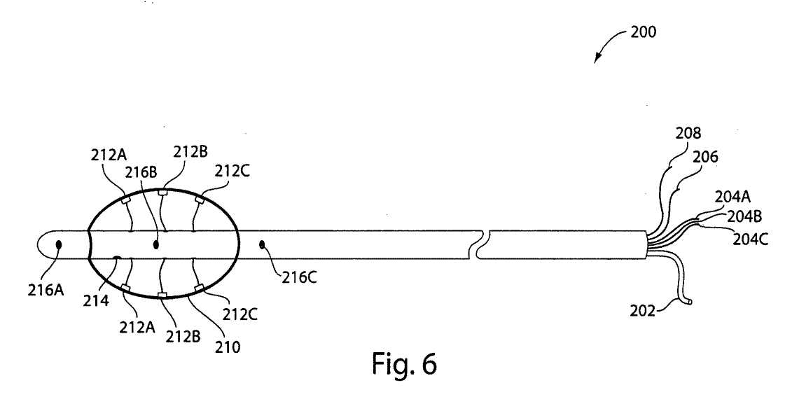

Referring to FIG. 6, with further reference to FIGS. 1 and 2, in an alternative embodiment of the smart balloon catheter system 10, the distal end of a smart balloon catheter 200 includes a saline transfer tube 202, ultrasound control lines 204a, 204b, 204c, electronic field control connection 206, transducer control connection 208, an inflatable membrane 210, three pairs of ultrasonic crystals 212a, 212b, 212c, a membrane saline port 214, and pressure sensors 216a, 216b, 216c. The pairs of ultrasonic crystals 212a, 212b, 212c are affixed to the interior side of the inflatable membrane 210, and are operably connected to an ultrasonic generator the ultrasound control lines 204a, 204b, 204c. The pairs of ultrasonic crystals 212a, 212b, 212c are configured to produce a signal which is proportional to the distance between each crystal in the pair. The computer 14 and monitor 16 are configured to acquire, calculate and display information corresponding to the volume enclosed by the inflatable membrane 210. For example, the functionality of the ultrasonic system is comparable to the Sonometrics SonoLab software system.

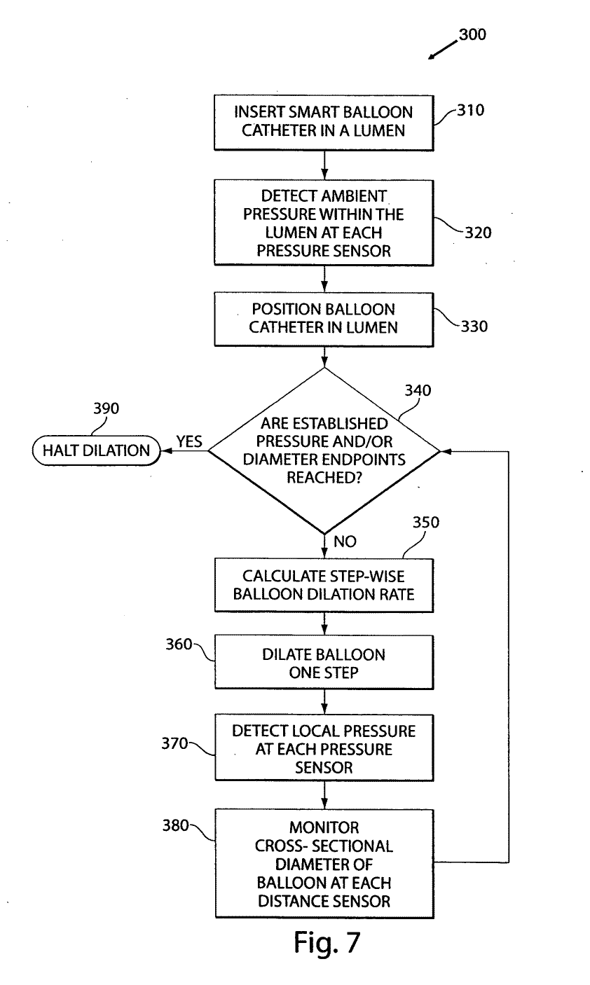

In operation, referring to FIG. 7, with further reference to FIGS. 1-6, a process 300 for the controlled dilation of a smart balloon catheter using the smart balloon catheter system 10 includes the stages shown. The process 300, however, is exemplary only and not limiting. The process 300 may be altered, e.g., by adding, removing, or rearranging stages.

At stage 310 the distal end of a smart balloon catheter 30 is inserted into a lumen 62. In general, the lumen 62 may include an obstruction 64 or other concretion 84 which have been identified via prior radiological or endoscopic observations. The shape and dilation performance of the inflatable membrane 32 on the catheter 30 can be selected based on the nature of the lumen 62, as well as the etiology of the obstruction 64 or the concretion 84.

At stage 320, an array of pressure transducers 34a, 34b, 34c detect the ambient pressure within the lumen 64. The computer 14 and monitor 18 receive, process, and display the pressure information simultaneously from each transducer. In one embodiment, the pumping system 22 provides a fluid to the inflatable membrane 32 to establish a set-point. For example, the set-point may represent the dilation of the inflatable membrane 32 to point the membrane 32 makes an initial physical contact with the walls of the lumen 62, without a significant change in pressure within the membrane 34b (i.e., the diameter of the membrane correlates with the diameter of the lumen).

At stage 330, the position of the catheter 30 is adjusted laterally within the lumen 62. The pressure sensors 34a, 34b, 34c provide features of a manometric catheter to localize the an area of high pressure such as in traversing a sphincter or narrowing. The position of the catheter 30 may also be established base on the diameter measurements detected by the impedance planimeters 38a, 38b, 38c. For example, differences in lumen diameter assists in positioning the membrane 32 within the obstruction 64. The catheter 30 can also be placed with fluoroscopic assistance, using the radio-opaque ring markers 48a, 48b to straddle the site of narrowing.

At stage 340, the computer 14 compares the information from the pressure sensors 34a, 34b, 34c, and the impedance planimeters 38a, 39b, 38c, with known pressure, volume and diameter endpoints. The endpoints are established and programmed prior to a controlled dilation of the membrane 30 and are dependent on etiology of the lumen 62 and the obstruction 64, as well as the type of membrane 30 used. The endpoints can be calculated on theoretical models, or can be based on empirical data, and are variables within the dilation feedback control algorithm. For example, endpoints can also be established within a dilation algorithm so that a rapid bougienage can be executed for mucosal strictures 126, or a more gradual dilation can be executed for muscular disorders 122. Also, a drop in pressure and/or equalization of diameters along the dilated membrane 30 (e.g., the fluoroscopic equivalent to eliminating the waist) can be used to terminate further dilation. If an establish endpoint is reached, the dilation is halted at step 390. Otherwise, the process continues to stage 350.

At stage 350, the computer 14 calculates the stepwise or controlled balloon dilation rate. The dilation rate is proportional to the amount of fluid pumped through the port 36 over a period of time. The computer 14 implements standard proportional and integral control logic to determine both the volume and rate in which the membrane 30 is filled with fluid.

At stage 360, the computer 14 sends a command to the pumping system 22 to provide fluid (e.g., from the reservoir 20 or the drip bag 24) to the membrane 30 at the controlled, or stepwise, rate determined at stage 350. For example, the pumping system 22 can provide a liquid via a positive displacement pump. Also, if the fluid is a gas, the pumping system 22 will deliver a steady flow of gas via a mass flow controller.

At stage 370, the pressure sensors 34a, 34b, 34c provide information to the computer 14. The sample rate of the pressure information is likely to be higher than the dilation rate delivered at stage 350, and therefore pressure information will arrive concurrently during dilation. Accordingly, the detection of the pressure endpoints at stage 340 is not limited to the completion of a full dilation step. The dilation can be halted at stage 340 before a dilation step is completed if a pressure related endpoint is reached.

At stage 380, the cross-sectional diameter of the membrane 30 is determined at each impedance planimeter 38a, 38b, 38c. The sampling rate of the distance information can be determined concurrently with the sampling of the pressure information and the dilation step. Accordingly, the detection of the distance and volume endpoints at stage 340 is not limited to the completion of a full dilation step. The dilation can be halted at stage 340 before a dilation step is completed if a distance/volume related endpoint is reached.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. A catheter system, comprising:
a distal pressure transducer;
a proximal pressure transducer;
an expandable membrane disposed between the distal pressure transducer and the proximal pressure transducer; and
a membrane pressure transducer disposed on the catheter within an area enclosed by the membrane, wherein the distal and proximal pressure transducers are configured to measure pressure outside of the membrane, and the membrane pressure transducer is configured to measure pressure inside of the membrane.

2. The catheter of claim 1 further comprising at least one barostat port disposed within the membrane.

3. The catheter of claim 2 wherein the membrane includes at least one radio-opaque marker configured to expand and contract with the membrane.

4. The catheter of claim 1 further comprising at least one pair of ultrasonic crystals affixed to the membrane.

5. A catheter for the diagnosis and treatment of a narrowing lumen, comprising:
a plurality of pressure transducers disposed within the catheter and arranged proximally from substantially near the distal end of the catheter;
at least one expandable membrane disposed over at least one pressure transducer;
at least one dilation source configured to expand the at least one membrane with a fluid; and
three pairs of electrode rings disposed proximally, center, and distally within the at least one membrane, wherein the electrode rings are configured to detect the diameter of the balloon membrane.

6. The catheter of claim 5 wherein the fluid is isotonic saline.

7. The catheter of claim 5 wherein the pressure transducers include thermoplastic elastomers (TPE).

8. A method for the diagnosis and assessment of a narrowing lumen with a balloon catheter, wherein the balloon catheter includes a plurality of pressure transducers, a balloon membrane, and a plurality of ring electrodes configured to detect the diameter of the lumen and the balloon membrane, the method comprising:
detecting ambient pressure within the lumen;
positioning the balloon catheter within the lumen;
dilating the balloon membrane; and
detecting the diameter of the balloon membrane.

9. The method of claim 8 further comprising detecting the diameter of the lumen.

10. The method of claim 8 further comprising executing a rapid dilation algorithm to perform a rapid bougienage on a mucosal stricture within the lumen.

11. The method of claim 8 further comprising executing a gradual dilation algorithm for muscular disorders within the lumen.

12. The method of claim 8 wherein the detecting the diameter of the balloon membrane includes detecting a plurality of diameters along the length of the balloon membrane.

13. The method of claim 12 further comprising terminating the dilation of the balloon catheter based on the equalization of diameters along the balloon membrane.

14. The method of claim 8 wherein the positioning of the balloon catheter within the lumen is based on the ambient pressure within the lumen.

15. The method of claim 8 further comprising detecting the pressure within the balloon membrane.

16. The method of claim 8 further comprising establishing an end-point for dilation of the balloon catheter based the diameter of the balloon membrane.

17. The method of claim 8 further comprising sweeping a concretion with the dilated balloon membrane.

18. A catheter comprising:
a balloon membrane;
inflation means coupled to the balloon membrane for controlled inflation of the balloon membrane;
sensing means disposed within the balloon catheter for simultaneously detecting the diameter of the balloon membrane and the pressure within the balloon membrane; and
processing means coupled to the inflation means and the sensing means for determining a rate of the controlled inflation of the balloon membrane.

19. The balloon catheter of claim 18 wherein the processing means is further configured for determining an end-point for the inflation of the balloon membrane.

20. The balloon catheter of claim 18 wherein the processing means is further configured to determine the rate of the controlled inflation with a closed loop proportional control algorithm.

21. A catheter comprising:
a balloon membrane;
at least one pair of ultrasonic crystals attached to the balloon membrane;
an ultrasound generator that is operably connected to the ultrasonic crystals and that is configured to determine the diameter of the balloon membrane;
a pressure transducer disposed on the balloon catheter and that is configured to sense the pressure within the balloon membrane; and
a radio-opaque marker disposed on the balloon membrane.

22. The balloon catheter of claim 21 wherein the balloon membrane is comprised of an elastic polymer.

23. The balloon catheter of claim 21 wherein the balloon membrane is a non-compliant balloon.

24. The balloon catheter of claim 21 wherein the balloon membrane is a compliant balloon.
